(19) United States
(12) Patent Application Publication
(10) Pub. No.: US 2023/0320845 A1
(43) Pub. Date: Oct. 12, 2023

(54) FLARED PROSTHETIC CARDIAC VALVE DELIVERY DEVICES AND SYSTEMS

(71) Applicant: SHIFAMED HOLDINGS, LLC, Campbell, CA (US)

(72) Inventors: Ali SALAHIEH, Saratoga, CA (US); Connor MULCAHY, San Francisco, CA (US); Alice YANG, Campbell, CA (US); Savina BRACHTAUSER, Los Gatos, CA (US)

(21) Appl. No.: 18/043,519

(22) PCT Filed: Aug. 31, 2021
(86) PCT No.: PCT/US2021/048478

§ 371 Date: Feb. 28, 2023

Related U.S. Application Data
(60) Provisional application No. 63/072,813, filed on Aug. 31, 2020.

(51) Int. Cl. A61F 2/24

(52) U.S. Cl. CPC A61F 2/2418; A61F 2250/0039; A61F 2250/001; A61F 2230/0091; A61F 2220/0016; A61F 2230/005; A61F 2210/0014; A61F 2002/075

(57) ABSTRACT

A device for treating a diseased native valve includes a frame structure having an unexpanded configuration and an expanded configuration, a plurality of leaflets coupled to the frame structure, and a spiral anchor configured to extend around the central annular portion. The frame structure in the expanded configuration includes a flared atrial portion, a central annular portion, and a flared ventricular portion. The frame structure includes a plurality of expandable cells and a plurality of paddles. Each of the paddles extends axially from a vertex of one of the plurality of expandable cells.

### CROSS-REFERENCE TO RELATED APPLICATIONS

This application is related to U.S. Provisional Patent Application No. 63/072,813, filed on Aug. 31, 2020, titled “FLARED PROSTHETIC CARDIAC VALVE DEVICES AND SYSTEMS,” the entirety of which is incorporated by reference in its entirety.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transeptally, requiring transapical access instead. Furthermore, existing mitral valve replacement devices are not optimized with respect to strength-weight ratio and often take up too much space within the valve chambers, resulting in obstruction of outflow from the ventricle into the aorta and/or thrombosis.

Thus, a new valve device that overcomes some or all of these deficiencies is desired.

### SUMMARY OF THE DISCLOSURE

In general, in one embodiment, a device for treating a diseased native valve includes a frame structure having an unexpanded configuration and an expanded configuration, a plurality of leaflets coupled to the frame structure, and a spiral anchor configured to extend around the central annular portion. The frame structure in the expanded configuration includes a flared atrial portion, a central annular portion, and a flared ventricular portion. The frame structure includes a plurality of expandable cells and a plurality of paddles. Each of the paddles extends axially from a vertex of one of the plurality of expandable cells.

This and other embodiments can include one or more of the following features. The device can further include a skirt positioned around an external surface of the frame structure. The skirt can include a plurality of tabs configured to cover an external surface of each of the paddles. Each of the plurality of paddles can include a linear extension and an eyelet. The linear extension can be non-foreshortening and the plurality of expandable cells can be foreshortening as the frame structure transitions from the unexpanded configuration to the expanded configuration. Each of the paddles can be curved so as to extend radially away from the vertex and then to point axially in an atrial direction. The expandable cells of the plurality of expandable cells can be arranged in a plurality of annular rows. The plurality of annular rows can include three annular rows. The frame structure can have an axial length of less than 35 mm in the expanded configuration. The spiral anchor can be a flat spiral anchor. The frame structure can be configured to self-expand from the unexpanded configuration to the expanded configuration. The frame structure can include nitinol. Each of the expandable cells can be substantially diamond shaped when the frame is in the expanded configuration. The flared atrial portion can be configured to extend further radially outwards than the flared ventricular portion. Every other vertex of the flared atrial portion can include a paddle of the plurality of paddles extending therefrom. The device can further include a plurality of tabs, and each tab can extend from a paddle of the plurality of paddles. The tabs can be configured to engage with a valve delivery system. The device can further include a webbing configured to extend along a ventricular end of the frame structure. The webbing can include a suture. The webbing can include a skirt. Expandable cells of the plurality of expandable cells can be smaller at an atrial end of the frame structure than at a ventricular end of the frame structure. The device can further include an atrial skirt on the flared atrial portion and a ventricular skirt on the flared ventricular portion. The atrial skirt can be woven, and the ventricular skirt can be knitted.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a valve frame.

FIG. 2A shows a valve frame with paddles.

FIG. 2B shows the flattened pattern of the valve frame of FIG. 2A.

FIG. 3A shows the stiffness profile of two different valve frames where the y-axis is the stiffness and the x-axis is the distance along the frame from the ventricular end to the atrial end.

FIG. 3B shows a valve frame with paddles where the valve frame is expanded against an anchor.

FIG. 3C shows a valve frame without paddles where the valve frame expanded against an anchor.

FIG. 4 shows a valve frame with paddles and an exterior atrial skirt and ventricular webbing.

FIG. 5A shows another embodiment of a valve frame with paddles.

FIG. 5B shows another embodiment of a valve frame with paddles.

FIGS. 6A-6B shows a valve frame with paddles, outer atrial and ventricular skirts, and an inner ventricular skirt.

### DETAILED DESCRIPTION

Described herein are systems, devices, and methods for treatment or replacement of a diseased native valve of the heart, such as the mitral valve.

In general, described herein is a replacement prosthesis that can include a valve frame and a spiral anchor therearound.

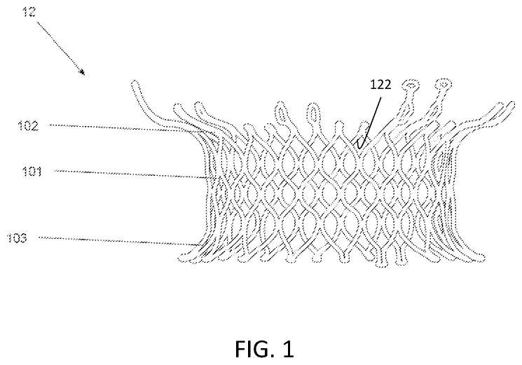

FIG. 1 shows a valve frame 12 in an expanded configuration. The valve frame 12 can be configured to hold a plurality of leaflets therein. Further, the valve frame 12 (and leaflets therein) can be deployed from a collapsed configuration to an expanded configuration during a method of replacing or repairing a mitral valve. As shown in FIG. 1, the valve frame 12 can include a plurality of rows (e.g., 3-7 rows) of substantially diamond-shaped cells 122. The valve frame 12 can foreshorten during delivery (i.e., as the valve frame 12 transitions from the collapsed configuration to the expanded configuration) due to the structure of the cells 122. In some embodiments, the valve frame 12 can be configured to self-expand from the collapsed configuration to the expanded configuration (e.g., can be made of nitinol).

One or more portions of the valve frame 12 can be shaped or configured to aid in securing the valve frame 12 at a location (e.g., in the orifice of a native heart valve). For example, the valve frame 12 can include an atrial flared portion 102 and a ventricular flared portion 103 configured to help secure the frame in the anatomy. The atrial and ventricular flared portions 102, 103 can extend radially outwards from a central circumferential portion 101. The atrial flared portion 102 can, for example, extend into the atrium of the heart from the central circumferential portion 101 when the valve prosthesis is deployed in the native mitral valve. The ventricular flared portion 103, in turn, can extend into the ventricle of the heart from the central circumferential portion 101 when the valve prosthesis is deployed in the native mitral valve. The atrial and ventricular flared portions 102, 103 can, for example, be configured to be positioned on either side of an external flat spiral anchor (e.g., that is wrapped around the chordae) to anchor the valve frame 12 in the anatomy. Alternatively or additionally, the atrial and ventricular flared portions 102, 103 can be configured to engage with tissue to prevent the valve prosthesis from slipping through the native valve orifice.

As shown in FIG. 1, the atrial flared portion 102 can extend further radially than the ventricular flared portion 103. Having a larger atrial flared portion can help prevent PVL.

In some embodiments, the longitudinal length of the collapsed valve frame 12 can be minimized, which can be advantageous for delivery of the valve frame 12. For example, minimizing the overall longitudinal length of the collapsed valve frame 12 can allow improved maneuverability within a delivery device while maintaining structural strength of the device. In some cases, minimizing the overall longitudinal length of the collapsed valve frame 12 can allow insertion of valve frame 12 through an access path that would be challenging for a longer device to traverse (e.g., an access path comprising tortuous passages or passages with sharp turns). In some cases, the valve frame 12 in the unexpanded configuration has an overall longitudinal length of from 1 mm to 50 mm, from 1 mm to 45 mm, from 1 mm to 40 mm, from 1 mm to 35 mm, from 1 mm to 30 mm, from 1 mm to 25 mm, from 1 mm to 20 mm, from 1 mm to 10 mm, from 10 mm to 45 mm, from 20 mm to 45 mm, from 20 mm to 30 mm, from 25 mm to 35 mm, or from 27.5 mm to 32.5 mm. In some cases, the valve frame 12 in the expanded configuration can have an overall longitudinal length of from 1 mm to 45 mm, from 10 mm to 45 mm, from 15 mm to 45 mm, from 15 mm to 35 mm, from 16 mm to 34 mm, from 17 mm to 33 mm, from 18 mm to 32 mm, from 19 mm to 31 mm, from 20 mm to 30 mm, from 25 mm to 35 mm, or from 27.5 mm to 32.5 mm. In some embodiments, the valve frame 12 can foreshorten as it expands such that the length in the expanded configuration is less than the length in the collapsed configuration.

In some embodiments, the valve frame 12 and/or overall prosthesis can have specific features designed to increase stiffness, improve control over valve deployment, promote uniform radial expansion of the central circumferential portion, ensure anchoring within the annulus, and/or decrease PVL.

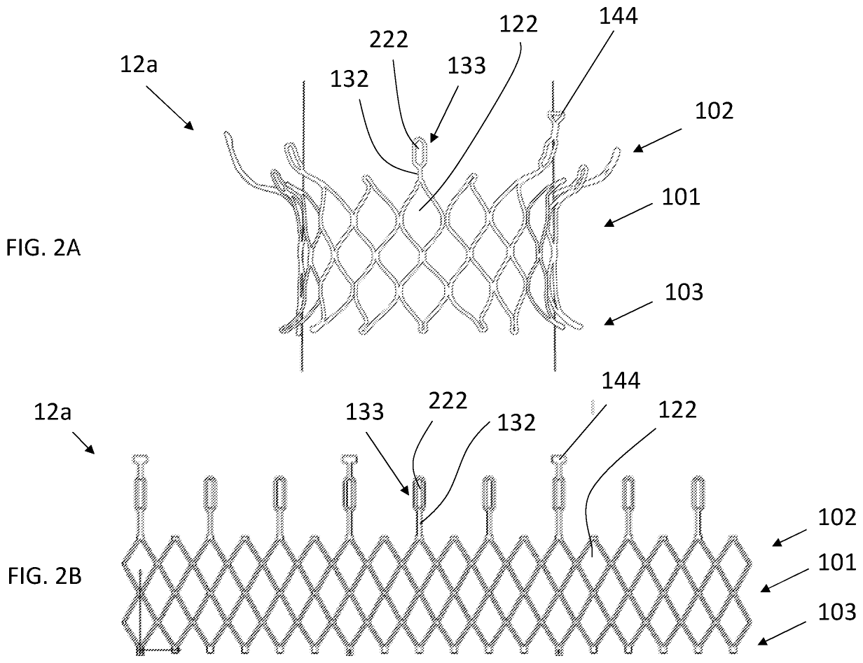

FIGS. 2A-2B show an exemplary frame 12a. The frame 12a can include a plurality (e.g., three) rows of cells 122. The cells 122 can be substantially diamond shaped and can be configured to foreshorten during deployment. Additionally, the atrial flared portion 102 of frame 12a can include a plurality of paddles 133 extending from a vertex (i.e., from the atrial tips) of some or all of the atrial cells 122 (for example, from every other vertex as shown in FIGS. 2A-2B). Each paddle can include a linear extension 132 and an eyelet 222. The paddles 133 can be configured to remain above (or proud of) other portions of the atrial flared portion 102 when implanted. In some embodiments, the paddles 133 can be configured to curve so as to extend radially outwards and then point at last partially in the axial (atrial) direction.

The linear extensions 132 of the paddles 133 can be non-foreshortening during deployment, which can advantageously help with trackability and sheathing of the atrial flared portion 102 during delivery of the frame 12a. The non-foreshortening extensions 132 can additionally advantageously provide increased stiffness and/or apposition of the atrial flared portion 102, thereby helping to better anchor the frame 12a in the anatomy. In some embodiments, because the valve frame 12a includes paddles 133, the axial length of the diamond-shaped cells 122 can be decreased while maintaining the same overall valve length. The shorter diamond-shaped cells can contribute to the increased stiffness of the valve frame 12a. The eyelets 222 can be used to affix (e.g., via suture) a skirt portion to the frame 12. Further, the eyelets 222 can advantageously enable grasping and/or manipulation of the valve frame 12a during deployment (for example, the eyelets 222 can be grasped with a hook or suture to enable controlled delivery, repositioning, or recapture of the frame 12a).

In some embodiments, the frame 12a can further include a plurality of (e.g., three) frame tabs 144 extending from the eyelets 222 and configured to engage with a valve delivery system for deployment. In one embodiment, each of the frame tabs 144 can be spaced apart by 120°.

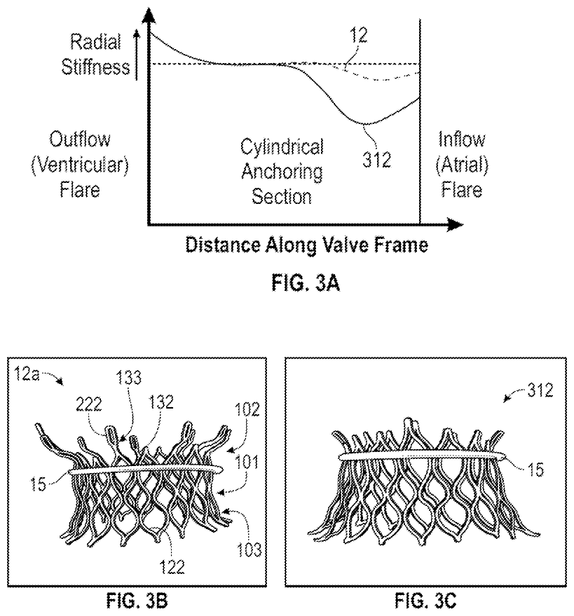

FIGS. 3A-3C show a comparison of the stiffness and expansion of the valve frame 12a with paddles 133 relative to a valve frame 312 without the paddles 133. For example, FIG. 3A shows the radial stiffness profile of valve frame 12a relative to valve frame 312. As shown in FIG. 3A, the stiffness of the valve frame 12a at the atrial end is much higher than the stiffness of the valve frame 312 at the at the atrial end, which can advantageously help anchor the valve frame 12a in the anatomy. The increased stiffness of the atrial portion 102 of the valve frame 12a can additionally ensure (as shown in the difference between FIGS. 3B and 3C) that the valve frame 12a is expanded more evenly from the atrial to the ventricular end (e.g., because the frame 12a can push against the anchor 15). This even expansion can advantageously ensure proper opening and closing of the leaflets and/or can enhance sealing of the valve prosthesis with the native annulus. Additionally, the increased stiffness of the atrial portion 102 of the frame 12a can advantageously hold the anchor 15 up higher towards the annulus, ensuring proper anchoring of the valve prosthesis.

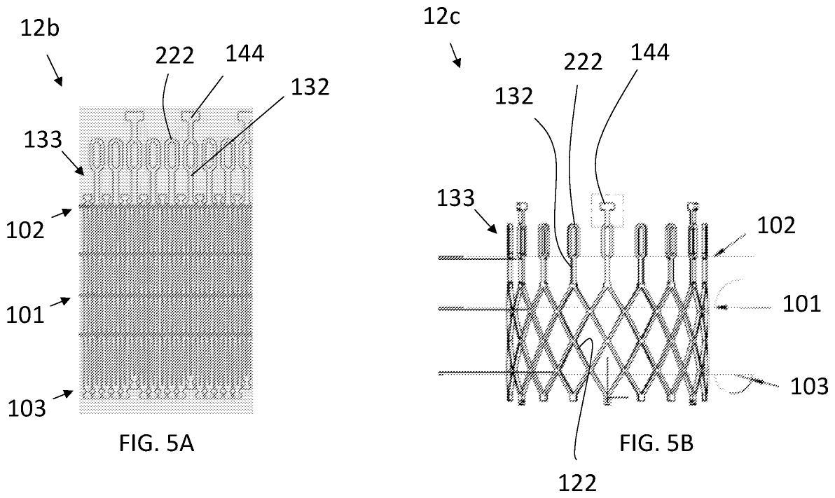

Additional variations of frame 12 are possible. For example, FIG. 5A shows a valve frame 12b that is similar to valve frame 12a, but that includes four rows of cells 122 rather than three. Additionally, the valve frame 12b includes a shorter ventricle cell 122 at each commissure location (e.g., to increase the stiffness of the cells 122 at the commissures to reduce pulsatile strain as the leaflets open and close). FIG. 5B shows a frame 12c that is similar to frame 12a except that frame 12c includes a paddle 133 connected to every atrial vertex rather than every other vertex.

In some frame embodiments, the cells 122 in and/or proximate to the atrial flared portion 102 can be smaller than the cells in the central circumferential portion 101 or the ventricular flared portion 103. Further, in some embodiments, the extensions 132 of the paddles 133 can include at least two inflection points, which can help orient the paddles 133 of the atrial flared portion 102 away from the anatomy, thereby making the paddles 133 less traumatic. Additionally or alternatively, ends of the paddles 133 may be rounded or curved to be made atraumatic.

Any of the valve frames 12 (e.g., frame 12a-12c) described herein can include one or more skirts or seals thereon. For example, the valve frame 12 can include an internal skirt and/or an external skirt. The internal or external skirt(s) can include a woven fabric, a knitted fabric, or a combination thereof. The fabric can include a biocompatible material such as a polyester, or polyethylene terephthalate (PET). The internal or external skirt(s) can comprise one or more coatings, for example a coating include a urethane (e.g., Chronoflex).

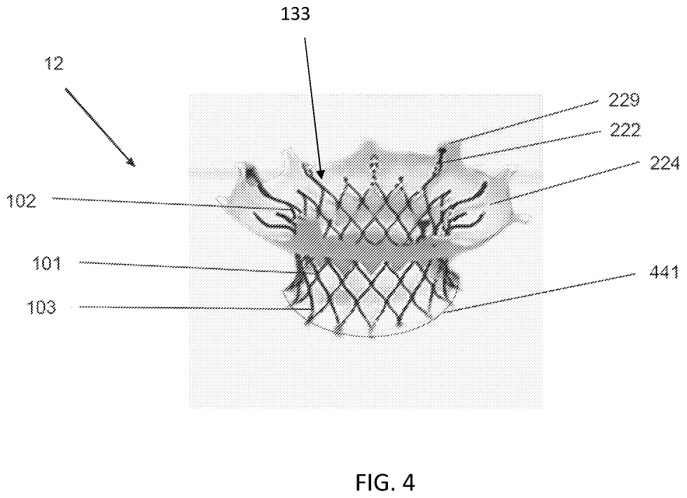

Referring to FIG. 4, in one embodiment, the atrial flared portion 102 of a valve frame 12 can have an atrial external skirt 224 formed therearound. The atrial external skirt 224 can be configured to be cut to conform to and/or closely mimic the shape of the paddles 133. Thus, for example, the atrial external skirt 224 can include skirt tabs 229 configured to cover the external portion of the paddles 133. In some embodiments, the skirt tabs 229 can be sewn to the paddles 133 at the eyelets 222. The atrial external skirt 224 can advantageously cover the gaps between the commissures that can occur when deploying a largely circular anchor valve into the D-shaped native valve. The atrial external skirt 224 can additionally or alternatively promote ingrowth, which can assist in anchoring the valve 12. In some embodiments, the atrial external skirt 224 can extend to the central circumferential portion 101 and/or the ventricular flared portion 103 as well. Referring still to FIG. 4, in some embodiments, the ventricular flared portion 103 can further include a webbing thereon. The webbing can include, for example, a suture line 441 and/or an external skirt. The webbing can advantageously prevent chordae from getting stuck in the cells and/or tips of the ventricular flared portion 103 and/or can aid in making the ventricular flared portion 103 less traumatic.

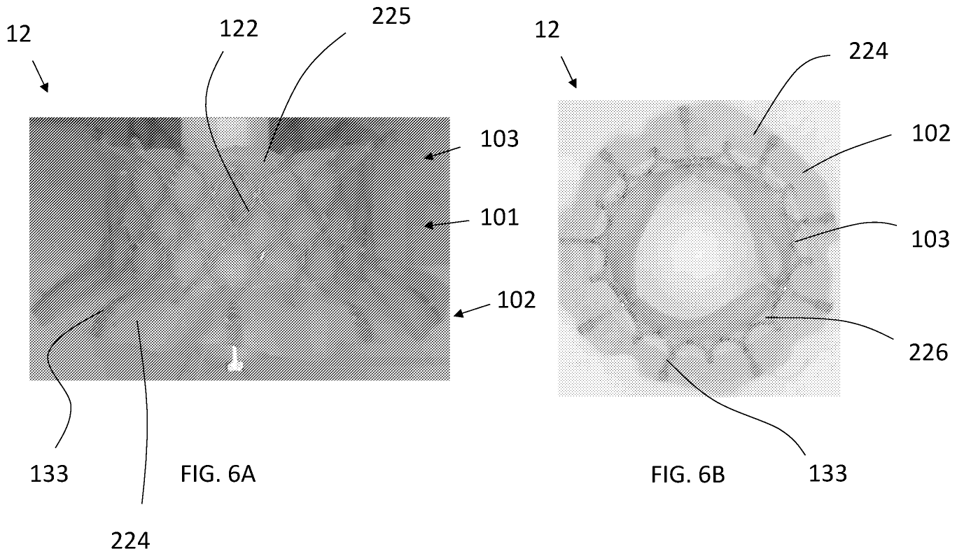

Referring to FIGS. 6A and 6B, in another embodiment, the atrial flared portion 102 of valve frame 12 can include an atrial external skirt 224, and the ventricular flared portion 103 can include both a ventricular external skirt 225 and a ventricular inner skirt 226. The atrial external skirt 224 and ventricular external skirt 225 can join and/or overlap with one another along the external surface of the valve frame 12. In some embodiments, skirt(s) on the atrial flared portion 102 (e.g., the atrial external skirt 224) can be made of a woven material while the skirt(s) on the ventricular flared portion 103 (e.g., the ventricular external skirt 225 or the ventricular inner skirt 226) can be made of a knitted material. Advantageously, the woven skirt may have less elongation and thereby match well with the nonforeshortning paddles 133 while the knitted skirt may have a greater elongation and thereby match well with the foreshortening cells 122.

The valve frame 12 may hold leaflets therein. In some embodiments, the leaflets can be formed of multi-layered materials for preferential function. The leaflets may be attached directly to the valve frame 12. Alternatively, the leaflets may be attached to an intermediate valve structure that is in turn connected to the valve frame 12. The leaflets may be connected to the valve frame 12 before or after the frame structure has been deployed adjacent a native valve. The leaflets may comprise a biocompatible one-way valve. Flow in one direction may cause the leaflets to deflect open and flow in the opposite direction may cause the leaflets to close. The valve frame 12 may be configured like a stent. The valve frame 12 may, for example, comprise a scaffold in a diamond pattern formed from a shape memory material (e.g., nitinol, NiTi). One of ordinary skill in the art will appreciate that many other structures, materials, and configurations may be employed for the frame structure. For example, valve frame 12 may be formed of a polymer of sufficient elasticity. The valve frame 12 may be formed of a combination of metal and polymer, such as metal (e.g., shape memory material) covered in polymer. The valve frame 12 may include a variety of patterns besides diamond shapes. In some embodiments, the valve frame 12 may be a closed frame such that blood flow is forced through the leaflets therein. One or more skirts and/or seals may help force blood through the leaflets. Exemplary valve frames and valve prostheses are described in PCT Application No. PCT/US2019/047542, filed on Aug. 21, 2019, titled “PROSTHETIC CARDIAC VALVE DEVICE, SYSTEMS, AND METHODS,” now PCT Publication No. WO 2020/041495 in International Patent Application No. PCT/US2020/027744, filed on Apr. 10, 2020, titled “MINIMAL FRAME PROSTHETIC CARDIAC VALVE DELIVERY DEVICES, SYSTEMS, AND METHODS,” now PCT Publication No. WO 2020/210685, and in International Patent Application No. PCT/US2021/037661, filed on Jun. 16, 2021, titled “MINIMAL FRAME PROSTHETIC CARDIAC VALVE DELIVERY DEVICES, SYSTEMS, AND METHODS,” the entireties of which are incorporated by reference herein.

Additionally, in some embodiments, the valve prostheses described may include an anchor 15. The anchor 15 may include a flat spiral shape with a plurality of windings or loops spiraling radially outwards from a central point. The loops of the flat spiral shaped anchor 15 may be generally positioned within the same plane. The anchor 15 may be formed from a shape memory material (e.g., NiTi). The anchor 15 can be configured to extend around the chordae of the valve (e.g., the mitral valve) and around the valve frame 12 to hold the valve prosthesis in place. Flat spiral anchors are described in U.S. patent application Ser. No. 16/723,537, filed on Dec. 20, 2019, titled “PROSTHETIC CARDIAC VALVE DEVICES, SYSTEMS, AND METHODS,” now U.S. Publication No. US-2020-0261220-A1, the entirety of which is incorporated by reference herein.

The valve prostheses (e.g., the valve frames and/or anchors) described herein may be delivered via a delivery system. Exemplary delivery systems are described in International Application No. PCT/US2020/023671, filed on Mar. 19, 2020, titled “PROSTHETIC CARDIAC VALVE DEVICES, SYSTEMS, AND METHODS,” now PCT Publication No. WO 2020/191216, and in International Application No. PCT/US2021/040623, filed on Jul. 7, 2021, titled “VALVE DELIVERY SYSTEM,” the entireties of which are incorporated by reference herein.

It should be understood that any feature described herein with respect to one embodiment can be substituted for or combined with any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for treating a diseased native valve, the device comprising:
a frame structure having an unexpanded configuration and an expanded configuration, the frame structure in the expanded configuration having a flared atrial portion, a central annular portion, and a flared ventricular portion, further wherein the frame structure comprises a plurality of expandable cells and a plurality of paddles, each of the paddles extending axially from a vertex of one of the plurality of expandable cells;
a plurality of leaflets coupled to the frame structure; and
a spiral anchor configured to extend around the central annular portion.

2. The device of claim 1, further comprising a skirt positioned around an external surface of the frame structure.

3. The device of claim 2, wherein the skirt comprises a plurality of tabs configured to cover an external surface of each of the paddles.

4. The device of claim 1, wherein the each of the plurality of paddles includes a linear extension and an eyelet.

5. The device of claim 4, wherein the linear extension is non-foreshortening and the plurality of expandable cells are foreshortening as the frame structure transitions from the unexpanded configuration to the expanded configuration.

6. The device of claim 1, wherein each of the paddles is curved so as to extend radially away from the vertex and then to point axially in an atrial direction.

7. The device of claim 1, wherein the expandable cells of the plurality of expandable cells are arranged in a plurality of annular rows.

8. The device of claim 7, wherein the plurality of annular rows comprises three annular rows.

9. The device of claim 1, wherein the frame structure has an axial length of less than 35 mm in the expanded configuration.

10. The device of claim 1, wherein the spiral anchor is a flat spiral anchor.

11. The device of claim 1, wherein the frame structure is configured to self-expand from the unexpanded configuration to the expanded configuration.

12. The device of claim 1, wherein the frame structure comprises nitinol.

13. The device of claim 1, wherein each of the expandable cells is substantially diamond shaped when the frame is in the expanded configuration.

14. The device of claim 1, wherein the flared atrial portion is configured to extend further radially outwards than the flared ventricular portion.

15. The device of claim 1, wherein every other vertex of the flared atrial portion comprises a paddle of the plurality of paddles extending therefrom.

16. The device of claim 1, further comprising a plurality of tabs, each tab extending from a paddle of the plurality of paddles, the tabs configured to engage with a valve delivery system.

17. The device of claim 1, further comprising a webbing configured to extend along a ventricular end of the frame structure.

18. The device of claim 17, wherein the webbing comprises a suture.

19. The device of claim 17, wherein the webbing comprises a skirt.

20. The device of claim 1, wherein expandable cells of the plurality of expandable cells are smaller at an atrial end of the frame structure than at a ventricular end of the frame structure.

21-22. (canceled)
